(19) United States
(12) Patent Application Publication
(10) Pub. No.: US 2025/0011717 A1
(43) Pub. Date: Jan. 9, 2025

(54) METHOD AND SYSTEMS FOR SCALABLE CULTURE OF ENTERIC GLIA CELLS

(71) Applicant: Case Western Reserve University, Cleveland, OH (US)

(72) Inventors: Paul J. Tesar, Wickliffe, OH (US); Marissa Scavuzzo, Cleveland, OH (US); Katherine Letai, Cleveland, OH (US)

(21) Appl. No.: 18/734,897

(22) Filed: Jun. 5, 2024

Related U.S. Application Data
(60) Provisional application No. 63/506,760, filed on Jun. 7, 2023.

(51) Int. Cl. C12N 5/079; G01N 33/50

(52) U.S. Cl. CPC C12N 5/0622; G01N 33/5058; C12N 2501/115; C12N 2501/13; C12N 2533/32; C12N 2533/52

(57) ABSTRACT

The invention described herein provides compositions and methods to culture enteric glial cells, and the associated use of the cultured enteric glial cells.

### REFERENCE TO RELATED APPLICATION

The application claims priority to U.S. Provisional Application No. 63/506,760, filed on Jun. 7, 2023, the entire contents of which are expressly incorporated by reference herein.

### GOVERNMENT SUPPORT

This invention was made with government support under Grant No. R35NS116842, awarded by the National Institute of Health. The government has certain rights in the invention.

### BACKGROUND OF THE INVENTION

The enteric nervous system is a complex network of nerves and glia embedded within the gastrointestinal (GI) tract that when dysfunctional contributes to disease or death at all ages. While enteric neurons are the subject of extensive investigation, the overwhelming majority (>90%) of the enteric nervous system is composed of a unique and diverse group of under-studied enteric-specific glia, called enteric glial cells.

Enteric glial cells are highly plastic, fragile cells embedded within multiple layers of the intestine closely intertwined with other, more adherent cell types like muscle. These cells are notoriously difficult to culture and work with. Other than immortalized cell lines which fail to recapitulate normal cellular biology in a dish or other tissue culture setting, current protocols only allow for terminal culturing of cells in a mixed cellular environment (Schonkeren et al., The gut brain in a dish: Murine primary enteric nervous system cell cultures. Neurogastroenterol Motil 34, e14215 (2022)). In addition, current protocols require the addition of serum, which contains unknown components that may alter enteric glia state or identity.

Together, these obstacles prevent the scale needed for certain assays including high-throughput drug screening, or forward genetic screens.

The generation of a new method to isolate, enrich, and expand enteric glia to overcome these issues would greatly benefit the field of enteric glia and gastroenterology.

### SUMMARY OF THE INVENTION

One aspect of the invention provides a chemically-defined, serum-free, enteric glial cell culture medium, suitable for culturing enteric glial cells (such as primary enteric glial cells, as well as ES- or iPSC-derived enteric glial cells), said culture medium comprising: i) a serum-free basal medium (such as 1 part DMEM/F12 and 1 part Neurobasal medium), wherein said basal medium (1) is devoid of significant source of proteins, lipids, or growth factors, and/or (2) comprises sufficient energy source, nitrogen source, carbon source, amino acids, vitamins, and inorganic salts to support growth of mammalian enteric glial cells; ii) 1×B27 type supplement; iii) 1×N−2 MAX type media supplement; and, iv) a trophic factor that promotes enteric glial cell survival in culture, comprising FGF2, GDNF, and NGF; and, optionally, further comprising: v) an antimicrobial agent for primary cells (e.g., a broad-spectrum antibiotic formulation active against bacteria, mycoplasma, and fungi, such as Primocin® brand broad-spectrum antibiotic formulation), optionally further comprising an antibiotic (such as Penicillin-Streptomycin); and, vi) a defined, serum-free supplement that increases cloning efficiency and single-cell survival of human embryonic stem cells (ES cells) or induced pluripotent stem cells (iPS cells) (such as CloneR™); or RHO-kinase inhibitor (such as Y-27632).

Another aspect of the invention provides a method of culturing primary enteric glial cells, the method comprising: 1) isolating primary enteric glial cells from a mammalian enteric tissue (such as intestine) physically separated from muscle, mesentery, and pancreatic tissues, through collagenase digestion; 2) resuspending isolated primary enteric glial cells in the enteric glial cell culture medium of the invention; and, 3) plating the resuspended primary enteric glial cells on a surface coated with poly-ornithine primed laminin for culturing (e.g., to confluence, or for about 2 weeks, before splitting).

Another aspect of the invention provides a method for analyzing the effect of a stimulus on enteric glial cells, the method comprising contacting an enteric glial cell cultured in the enteric glial cell culture medium of the invention, and/or using the method of the invention, with the stimulus, and determining a readout.

Yet another aspect of the invention provides an enteric glial cell culture comprising a population of (primary) enteric glial cells cultured in the culture medium of the invention.

### BRIEF DESCRIPTION OF THE DRAWINGS

FIGS. 1A-1C show primary enteric glia dissection and media optimization. FIG. 1A shows microdissection of longitudinal muscle myenteric plexus (LMMP) layer of the intestine.

FIG. 1B shows immunostaining of LMMP for markers of glia (GFAP) and neurons (Tuj1) with nuclei marked by DAPI in blue. FIG. 1C shows time course of media compositions using transgenic Plp1-eGFP primary cells. Far left 10% FBS is current media composition used in the field; cells fail to expand. GDNF+FGF2 and GDNF+FGF2+BMP4 are media formulas of the invention.

FIGS. 2A-2E show functional assessment and further specification of media composition and ECM. FIG. 2A shows functional test of primary enteric glial cells for their response to cytokines. FIG. 2B shows representative immunostaining of cells shown quantified in FIG. 2A. GBP2 marks reactive enteric glia while Plp1-eGFP marks enteric glial cells currently expressing Plp1, and GFAP marks enteric glia. Nuclei are marked by DAPI in blue. FIG. 2C shows percent Plp1-cGFP positive cells after passaging. FIG. 2D shows further modification of media composition; addition of NGF to GDNF+FGF2 promotes eGFP+cell expansion and survival. FIG. 2E shows ECM array heatmap showing negative control on the bottom and several ECM compositions above. Cell number and enteric glial cell number are depicted in each column.

FIGS. 3A-3C show plating and sorting of enteric glial cells. FIG. 3A shows addition of RHO-kinase inhibitor or CloneR improves cell attachment. FIG. 3B are FACS plots showing sorting of eGFP⁺ cells and a post-sort purity check. FIG. 3C shows live imaging of Plp1-cGFP cells after sorting. Cells continue to grow and expand.

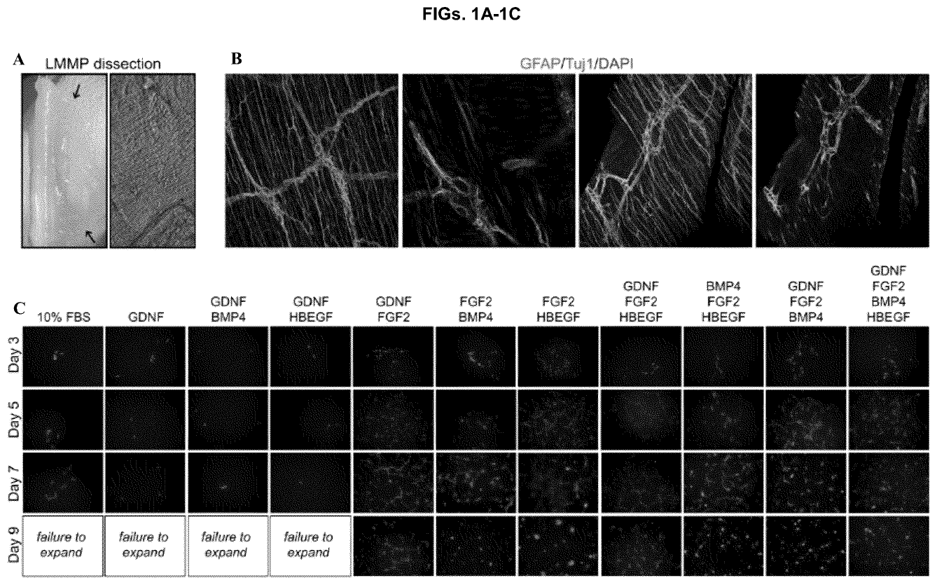

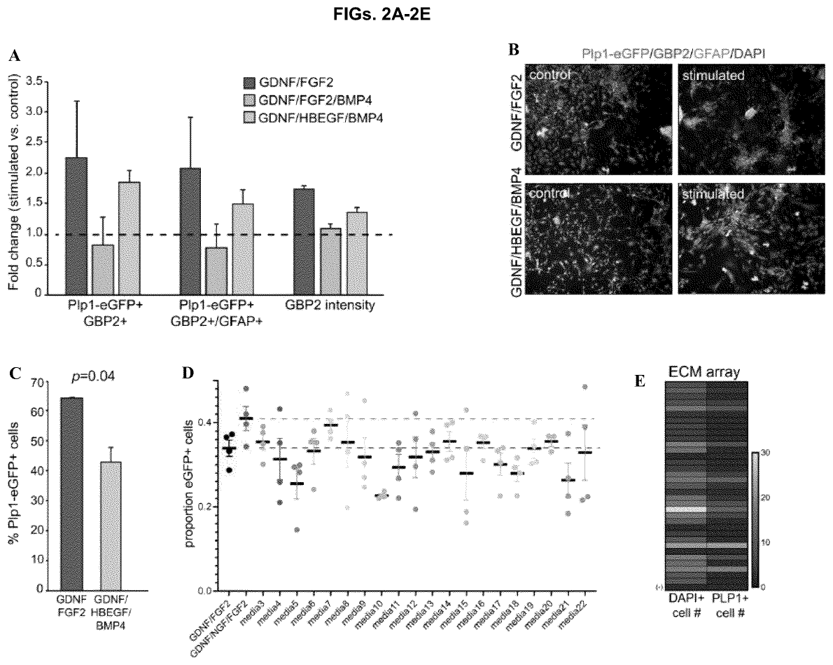

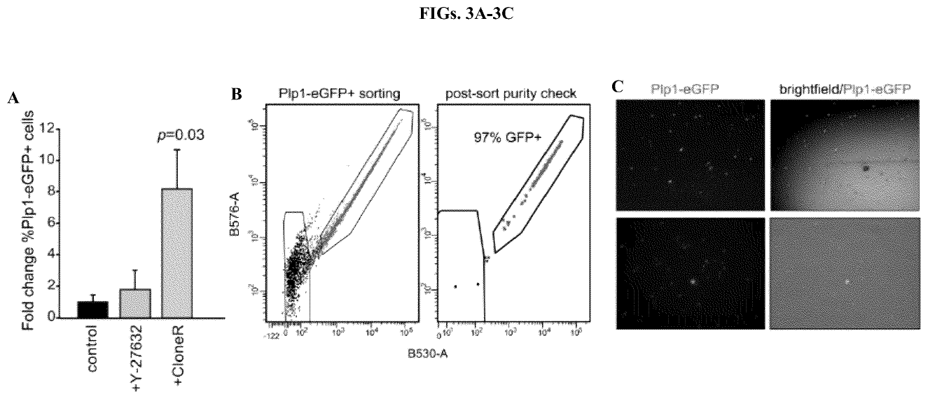

### DETAILED DESCRIPTION OF THE INVENTION

Described herein is a new technology to culture enteric glial cells, such as in a pure and scalable manner in defined media without the addition of serum. Numerous iterations of media formulations, extracellular matrix compositions, dissection methods, and attachment methods were tested in order to outline and identify specific combinations that provided the best growth and expansion of enteric glia.

In addition to using transgenic readouts of cell identity in real time via live imaging, functional readouts of enteric glial cell responses to environmental stimuli were also performed. This led to the invention described herein, which provides the first ever platform for large scale enteric glial cell culture to utilize for a wide range of applications.

Potential use of this technology includes high-throughput chemical or genetic screens. Enteric glia can be generated from mouse models of disease or treated with exogenous stressors to model enteric glia pathology. Due to the scalability of this technology, these in vitro models of pathology can be screened against chemical or genetic libraries, for example a library of siRNAs, to discover chemicals or genetic perturbations that reduce or promote enteric glia pathology. Enteric glia pathology or reactivity is a characteristic of many gastrointestinal and neurological diseases including but not limited to inflammatory bowel disease, obesity, Parkinson's disease, Alexander's disease, autism spectrum disorder, and schizophrenia. Enteric glia are also incredibly plastic and robustly respond to changes in their environment.

An exemplary use of this technology is the identification of diverse glial cell morphologies. Enteric glia have been shown to exhibit four different morphologies, however these studies were done through direct injection of HRP into individual cells in the myenteric plexus (Hanani and Reichenbach, Morphology of horseradish peroxidase (HRP)-injected glial cells in the myenteric plexus of the guinea-pig. Cell Tissue Res 278, 153-160 (1994)). For a high-throughput analysis of enteric glia morphology, we utilized this protocol and confirmed the four described enteric glia morphological subtypes, but also uncovered two additional subtypes. Using this high-throughput and scalable method, we split enteric glial cells into 96-well plates and performed multiplexed immunostainings to identify morphological subtypes exhibiting different molecular properties.

Thus in one aspect of the invention provides a chemically-defined, serum-free, enteric glial cell culture medium, suitable for culturing enteric glial cells (such as primary enteric glial cells, as well as ES- or iPSC-derived enteric glial cells), said culture medium comprising: i) a serum-free basal medium (such as 1 part DMEM/F12 and 1 part Neurobasal medium), wherein said basal medium (1) is devoid of significant source of proteins, lipids, or growth factors, and/or (2) comprises sufficient energy source, nitrogen source, carbon source, amino acids, vitamins, and inorganic salts to support growth of mammalian enteric glial cells; ii) 1×B27 type supplement; iii) 1×N-2 MAX type media supplement; and, iv) a trophic factor that promotes enteric glial cell survival in culture, comprising FGF2, GDNF, and NGF; and, optionally, further comprising: v) an antimicrobial agent for primary cells (e.g., a broad-spectrum antibiotic formulation active against bacteria, mycoplasma, and fungi, such as Primocin® brand broad-spectrum antibiotic formulation), optionally further comprising an antibiotic (such as Penicillin-Streptomycin); and, vi) a defined, serum-free supplement that increases cloning efficiency and single-cell survival of human embryonic stem cells (ES cells) or induced pluripotent stem cells (iPS cells) (such as CloneR™); or RHO-kinase inhibitor (such as Y-27632).

In certain embodiments, the glial cells are isolated primary enteric glial cells. In certain embodiments, the glial cells are ES- or iPSC-derived enteric glial cells.

In certain embodiments, the optional component(s) v) and/or vi) is/are absent. For example, component v) is not required for culturing the glial cells per se, but can be helpful for culturing, e.g., isolated primary cells from the gut where various microbes may be present in large quantity. Meanwhile, glial cells that derive from ES cells ir iPS cells may have less stringent requirements of having component v) in the medium, and may not need such component under certain conditions.

In certain embodiments, the serum-free basal medium comprises, consists essentially of, or consists of 1 part DMEM/F12 and 1 part Neurobasal medium.

In certain embodiments, the B27 type supplement is B27 supplement (e.g., TermoFisher B-27TM Brand B27 Supplement, Cat. No. 17504044).

In certain embodiments, the N-2 MAX type media supplement is N-2 MAX media supplement (e.g., R&D Systems N-2 MAX Media Supplement, Cat. No. AR009).

In certain embodiments, the trophic factor comprises about 5-20 ng/mL (e.g., about 10 ng/mL) GNDF, about 10-40 ng/mL (e.g., about 20 ng/mL) FGF2, and about 25-100 ng/mL (e.g., about 50 ng/mL) NGF.

In certain embodiments, the antimicrobial agent for primary cells is a broad-spectrum antibiotic formulation active against bacteria, mycoplasma, and fungi (such as 1×Primocin® brand broad-spectrum antibiotic formulation); optionally, the culture medium further comprises an antibiotics (such as 1x Pen/Strep).

In certain embodiments, the defined, serum-free supplement that increases cloning efficiency and single-cell survival of human ES cells or iPS cells is 1×CloneR™ brand supplement (e.g., Stemcell Technologies Cat. No. 05889).

In certain embodiments, the primary enteric glial cells are isolated from intestine, such as longitudinal muscle myenteric plexus (LMMP).

In certain embodiments, the primary enteric glial cells are isolated from a mammal, such as a human, a non-human primate (NHP such as monkey, ape, chimpanzee, gorilla), a rodent (e.g., mouse, rat, hamster, guinea pig, rabbit), a livestock mammal (e.g., cattle, cow, bull, sheep, goat, horse, or pig), or a pet (dog or cat).

Another aspect of the invention provides a method of culturing primary enteric glial cells, the method comprising: 1) isolating primary enteric glial cells from a mammalian enteric tissue (such as intestine) physically separated from muscle, mesentery, and pancreatic tissues, through collagenase digestion; 2) resuspending isolated primary enteric glial cells in the enteric glial cell culture medium of the invention; and, 3) plating the resuspended primary enteric glial cells on a surface coated with poly-ornithine primed laminin for culturing (e.g., to confluence, or for about 2 weeks, before splitting).

In certain embodiments, the method further comprises exchanging media once every two days, with the enteric glial cell culture medium of the invention without components v) and/or vi).

In certain embodiments, the surface is coated by 0.1 mg/mL poly-ornithine primed, 10 μg/mL laminin.

In certain embodiments, step 2) is performed in a digestive KREBS solution buffer comprising: about 0.6-2.6 mg/mL (e.g., about 1.3 mg/mL) collagenase type II (e.g., Worthington, LS004176), about 0.15-0.6 mg/mL (e.g., about 0.3 mg/mL) BSA (e.g., Sigma, A4161), about 60-245 mM (e.g., about 121 mM NaCl), about 3-12 mM (e.g., about 5.9 mM) KCl, about 1.25-5 mM (e.g., about 2.5 mM) CaCl₂), about 0.6-2.5 mM (e.g., about 1.2 mM) MgSO₄, about 0.6-2.4 mM (e.g., about 1.2 mM) NaH₂PO₄, about 5-20 nM (e.g., about 10 mM) HEPES, about 10.5-45 mM (e.g., about 21.2 mM NaHCO₃, and about 4-16 mM (e.g., about 8 mM) glucose.

In certain embodiments, step 2) is performed at about 37° C., bubbled with carbogen for about 1 hour.

In certain embodiments, the step 2) is performed within 30 minutes of completing step 1).

In certain embodiments, the primary enteric glial cells are isolated from a mammal, such as a human, a non-human primate (NHP such as monkey, ape, chimpanzee, gorilla), a rodent (e.g., mouse, rat, hamster, guinea pig, rabbit), a livestock mammal (e.g., cattle, cow, bull, sheep, goat, horse, or pig), or a pet (dog or cat).

In certain embodiments, the mammal is a model for a disease, or has been treated with an exogenous or environmental stressor to model enteric glia pathology.

In certain embodiments, the disease is a gastrointestinal and/or a neurological disease, such as inflammatory bowel disease, obesity, Parkinson's disease, Alexander's disease, autism spectrum disorder, or schizophrenia.

Another aspect of the invention provides a method for analyzing the effect of a stimulus on enteric glial cells, the method comprising contacting an enteric glial cell cultured in the enteric glial cell culture medium of the invention, and/or using the method of the invention, with the stimulus, and determining a readout.

In certain embodiments, the stimulus is a chemical compound (e.g., small molecule or macromolecule drug candidate), an exogenous or environmental stressor to a model of enteric glia pathology.

In certain embodiments, the enteric glial cell is isolated from a mammal, such as a human, a non-human primate (NHP such as monkey, ape, chimpanzee, gorilla), a rodent (e.g., mouse, rat, hamster, guinea pig, rabbit), a livestock mammal (e.g., cattle, cow, bull, sheep, goat, horse, or pig), or a pet (dog or cat).

In certain embodiments, the mammal is a model for a disease.

In certain embodiments, the disease is a gastrointestinal and/or a neurological disease, such as inflammatory bowel disease, obesity, Parkinson's disease, Alexander's disease, autism spectrum disorder, or schizophrenia.

Yet another aspect of the invention provides an enteric glial cell culture comprising a population of (primary) enteric glial cells cultured in the culture medium of the invention.

It should be understood that any one embodiment described herein, including those described only in the Example or claims, can be combined with any one or more additional embodiments of the invention unless expressly disclaimed or being improper.

### EXAMPLES

The following is an illustrative embodiment to highlight certain non-limiting features of the invention described herein, and thus should not be construed to limit the scope of the invention described herein in any respect.

### Example 1 Enteric Glial Cell Culture Medium and Culture Method

In brief, mouse intestinal tissue was dissected, flushed with PBS to remove luminal contents, and placed in PBS on ice.

Mesentery and pancreatic tissue were then removed, then intestinal tissue was cut into 1.0-1.5 cm segments. Tissue segments were individually slid onto a borosilicate glass Pasteur pipette. Using pressure from a finger for stability and under microscopy, forceps were used to create a thin nick along the serosa and muscle layers. A cotton swap is wetted with cold PBS and used to wipe along the nick while slowly turning the tissue using the stabilizing finger to peel away the longitudinal muscle and myenteric plexus layer.

Collected tissue was immediately placed in a digestive KREBS solution buffer with 1.3 mg/mL collagenase type II (Worthington, LS004176) and 0.3 mg/mL BSA (Sigma, A4161). The KREBS solution was composed of the following: 121 mM NaCl, 5.9 mM KCl, 2.5 mM CaCl₂), 1.2 mM MgSO₄, 1.2 mM NaH₂PO₄, 10 mM HEPES, 21.2 mM NaHCO₃, and 8 mM glucose. All tissues were collected and placed into the solution on ice within 30 minutes of dissection.

The digestive KREBS solution was then moved to 37° C., and bubbled with carbogen for 1 hour.

Following this hour, solution was quenched with base media, and was centrifuged at 356 g for 10 minutes at 4° C. The Enteric glial cell media composition used here is outlined below.

### Media Formulations and Culture Conditions-Plating

1:1 DMEM/F12+Neurobasal
1× Pen/Strep
1×N2-Max
1×B27, serum free
20 ng/mL FGF2
10 ng/mL GDNF
50 ng/mL NGF
50 g/mL primocin
1× CloneR

### Media Formulations and Culture Conditions—Maintenance

1:1 DMEM/F12+Neurobasal
1×Pen/Strep
1×N2-Max
1×B27, serum free
20 ng/mL FGF2
10 ng/mL GDNF
50 ng/mL NGF

Cells are plated/maintained on 0.1 mg/mL poly-ornithine primed, 10 μg/mL laminin 10 cm plates (2 mice per plate).

The remaining pellet was gently resuspended in enteric glial cell media supplemented with 1×CloneR (Stem Cell Technologies, 5889) and 50 g/mL primocin (Invivogen, ant-pm-1), filtered through 100 μM filters (Fisher Scientific, 08-771-19), and plated onto 10 cm 0.1 mg/mL poly-ornithine primed, 10 μg/mL laminin coated plates. Media was replaced every other day and cells were split using Accutase at room temperature after reaching confluence or 2 weeks in culture.

Enteric glia from Plp1-eGFP mice retain eGFP expression, enteric glia morphological diversity, and respond to cytokine stimulation by upregulating reactive glia markers.

1. A chemically-defined, serum-free, enteric glial cell culture medium, suitable for culturing enteric glial cells, said culture medium comprising:
i) a serum-free basal medium, wherein said basal medium (1) is devoid of significant source of proteins, lipids, or growth factors, and/or (2) comprises sufficient energy source, nitrogen source, carbon source, amino acids, vitamins, and inorganic salts to support growth of mammalian enteric glial cells;
ii) 1×B27 type supplement;
iii) 1×N-2 MAX type media supplement; and,
iv) a trophic factor that promotes enteric glial cell survival in culture, comprising FGF2, GDNF, and NGF; and,
optionally, further comprising:
v) an antimicrobial agent for primary cells, optionally further comprising an antibiotic; and,
vi) a defined, serum-free supplement that increases cloning efficiency and single-cell survival of human embryonic stem cells (ES cells) or induced pluripotent stem cells (iPS cells); or RHO-kinase inhibitor.

2. The enteric glial cell culture medium of claim 1, wherein the serum-free basal medium comprises, consists essentially of, or consists of 1 part DMEM/F12 and 1 part Neurobasal medium.

3. The enteric glial cell culture medium of claim 1, wherein the B27 type supplement is B27 supplement.

4. The enteric glial cell culture medium of claim 1, wherein the N−2 MAX type media supplement is N−2 MAX media supplement.

5. The enteric glial cell culture medium of claim 1, wherein the trophic factor comprises about 5-20 ng/mL GNDF, about 10-40 ng/mL FGF2, and about 25-100 ng/mL NGF.

6. The enteric glial cell culture medium of claim 1, wherein the antimicrobial agent for primary cells is a broad-spectrum antibiotic formulation active against bacteria, mycoplasma, and fungi; optionally, the culture medium further comprises an antibiotics.

7. The enteric glial cell culture medium of claim 1, wherein the defined, serum-free supplement that increases cloning efficiency and single-cell survival of human ES cells or iPS cells is 1×CloneR™ brand supplement.

8. The enteric glial cell culture medium of claim 1, wherein:
the primary enteric glial cells are isolated from intestine, such as longitudinal muscle myenteric plexus (LMMP); and/or
the primary enteric glial cells are isolated from a mammal, such as a human, a non-human primate (NHP), a rodent, a livestock mammal, or a pet.

9. (canceled)

10. A method of culturing primary enteric glial cells, the method comprising:
1) isolating primary enteric glial cells from a mammalian enteric tissue physically separated from muscle, mesentery, and pancreatic tissues, through collagenase digestion;
2) resuspending isolated primary enteric glial cells in the enteric glial cell culture medium of claim 1; and,
3) plating the resuspended primary enteric glial cells on a surface coated with poly-ornithine primed laminin for culturing.

11. The method of claim 10, further comprising exchanging media once every two days, with the enteric glial cell culture medium, wherein the enteric glial cell culture medium comprises:
i) a serum-free basal medium, wherein said basal medium (1) is devoid of significant source of proteins, lipids, or growth factors, and/or (2) comprises sufficient energy source, nitrogen source, carbon source, amino acids, vitamins, and inorganic salts to support growth of mammalian enteric glial cells;
ii) 1×B27 type supplement;
iii) 1×N−2 MAX type media supplement; and,
iv) a trophic factor that promotes enteric glial cell survival in culture, comprising FGF2, GDNF, and NGF; and,
optionally, further comprising:
v) an antimicrobial agent for primary cells, optionally further comprising an antibiotic; and/or,
vi) a defined, serum-free supplement that increases cloning efficiency and single-cell survival of human embryonic stem cells (ES cells) or induced pluripotent stem cells (iPS cells); or RHO-kinase inhibitor.

12. The method of claim 10, wherein the surface is coated by 0.1 mg/mL poly-ornithine primed, 10 μg/mL laminin.

13. The method of claim 10, wherein step 2) is performed in a digestive KREBS solution buffer comprising: about 0.6-2.6 mg/ml collagenase type II, about 0.15-0.6 mg/mL BSA, about 60-245 mM NaCl, about 3-12 mM KCl, about 1.25-5 mM CaCl₂, about 0.6-2.5 mM MgSO₄, about 0.6-2.4 mM NaH₂PO₄, about 5-20 nM HEPES, about 10.5-45 mM NaHCO₃, and about 4-16 mM glucose.

14. The method of claim 13, wherein step 2) is performed at about 37° C., bubbled with carbogen for about 1 hour.

15. The method of claim 10, wherein step 2) is performed within 30 minutes of completing step 1).

16. The method of claim 10, wherein:
the primary enteric glial cells are isolated from a mammal, such as a human, a non-human primate (NHP), a rodent, a livestock mammal, or a pet; and/or
the primary enteric glial cells are isolated from a mammal, wherein the mammal is a model for a disease, or has been treated with an exogenous or environmental stressor to model enteric glia pathology.

17. (canceled)

18. The method of claim 16, wherein the disease is a gastrointestinal and/or a neurological disease, such as inflammatory bowel disease, obesity, Parkinson's disease, Alexander's disease, autism spectrum disorder, or schizophrenia.

19. A method for analyzing the effect of a stimulus on enteric glial cells, the method comprising:
contacting an enteric glial cell cultured in the enteric glial cell culture medium, wherein the enteric glial cell culture medium comprises:
i) a serum-free basal medium (such as 1 part DMEM/F12 and 1 part Neurobasal medium), wherein said basal medium (1) is devoid of significant source of proteins, lipids, or growth factors, and/or (2) comprises sufficient energy source, nitrogen source, carbon source, amino acids, vitamins, and inorganic salts to support growth of mammalian enteric glial cells;
ii) 1×B27 type supplement;
iii) 1×N−2 MAX type media supplement; and,
iv) a trophic factor that promotes enteric glial cell survival in culture, comprising FGF2, GDNF, and NGF; and,
optionally, further comprising:
v) an antimicrobial agent for primary cells, optionally further comprising an antibiotic; and,
vi) a defined, serum-free supplement that increases cloning efficiency and single-cell survival of human embryonic stem cells (ES cells) or induced pluripotent stem cells (iPS cells); or RHO-kinase inhibitor, and/or
using the method of claim 10, with the stimulus, and determining a readout.

20. The method of claim 19, wherein:
the stimulus is a chemical compound (e.g., small molecule or macromolecule drug candidate), an exogenous or environmental stressor to a model of enteric glia pathology;
the enteric glial cell is isolated from a mammal, such as a human, a non-human primate (NHP), a rodent, a livestock mammal, or a pet; and/or
the enteric glial cell is isolated from a mammal, wherein the mammal is a model for a disease.

21-22. (canceled)

23. The method of claim 20, wherein the disease is a gastrointestinal and/or a neurological disease, such as inflammatory bowel disease, obesity, Parkinson's disease, Alexander's disease, autism spectrum disorder, or schizophrenia.

24. An enteric glial cell culture comprising a population of (primary) enteric glial cells cultured in the culture medium of claim 1.
